(19) United States
(12) Patent Application Publication
(10) Pub. No.: US 2016/0354413 A1
(43) Pub. Date: Dec. 8, 2016

(54) ENCAPSULATED CELLS FOR TREATING LOW TESTOSTERONE IN MALE SUBJECTS

(71) Applicant: WAKE FOREST UNIVERSITY HEALTH SCIENCES, Winston-Salem, NC (US)

(72) Inventor: Emmanuel C. Opara, Durham, NC (US)

(73) Assignee: Wake Forest University Health Sciences, Winston-Salem, NC (US)

(21) Appl. No.: 15/103,962

(22) PCT Filed: Dec. 15, 2014
(86) PCT No.: PCT/US2014/070314

§ 371 Date: Jun. 13, 2016

Related U.S. Application Data
(60) Provisional application No. 61/918,969, filed on Dec. 20, 2013.

(51) Int. Cl. A61K 35/54; A61K 35/48; A61K 9/50; A61K 35/52

(52) U.S. Cl. CPC A61K 35/54; A61K 35/52; A61K 35/48; A61K 9/5073; A61K 9/5036; A61K 9/5031

(57) ABSTRACT

A pharmaceutical composition for treating low testosterone comprises microcapsules the microcapsules containing live mammalian ovary cells. The ovary cells comprise ovarian theca cells in a treatment-effective amount, but not granulosa cells or without granulosa cells in amounts detrimental to the administration of testosterone. Methods of treating male subjects afflicted with low testosterone by administration of such ovary cell-containing microcapsules are also described.

### FIELD OF THE INVENTION

The present invention concerns compositions and methods for treating low testosterone in male subjects in need thereof.

### BACKGROUND OF THE INVENTION

Testosterone levels in men decrease as a natural consequence of aging, with the level of testosterone generally declining at a rate of one percent annually for each year over age 30. In 2013, the American Diabetes Association (ADA) estimated that over 13 million men have low testosterone levels.

Current treatments for low testosterone often rely on topical or oral testosterone administration. Handling these types of products on a regular basis risks exposure of others to testosterone, which may have undesired side-effects. Accordingly, there is a need for new ways to administer testosterone to subjects in need thereof.

### SUMMARY OF THE INVENTION

A first aspect of the present invention is a pharmaceutical composition for treating low testosterone, the composition comprising microcapsules, said microcapsules containing live mammalian ovary cells. The said ovary cells consist essentially of ovarian theca cells in a treatment-effective amount (that is, theca cells but not granulosa cells, or without granulosa cells in amounts detrimental to achieving the goal of administering testosterone, as discussed below).

The microcapsules may optionally also contain live mammalian Sertoli cells and/or live mammalian Leydig cells. The Sertoli cells, Leydig cells and theca cells may be contained in separate microcapsules in said composition, or together in the same microcapsules in the composition. For example, the microcapsules may comprise a core and an auxiliary layer surrounding said core; with the core containing one of said Sertoli cells and said theca cells and said auxiliary layer containing the other of said Sertoli cells and said theca cells; and with the core and/or said auxiliary layer optionally further containing said Leydig cells.

Methods of using the foregoing for the treatment of low testosterone are also described herein.

The present invention is explained in greater detail in the specification set forth below. The disclosures of all United States patent references cited herein are to be incorporated by reference herein in their entirety.

### DETAILED DESCRIPTION OF THE PREFERRED EMBODIMENTS

“Subjects” as used herein are male and, in general, mammalian, subjects. While human subjects are preferred, the subjects may in some embodiments be other animals, such as dogs and cats for veterinary purposes. While the subjects may be of any suitable age, the subjects are typically adults and in some embodiments are at least 40, 45, 50, 55 or 60 years of age.—Apart from subjects afflicted with low testosterone due to aging, subjects to be treated by the methods and compositions described herein may be afflicted with low testostereone levels due to injury, infection, or loss of the testicles; chemotherapy or radiation treatment for cancer; genetic abnormalities such as Klinefelter's Syndrome (extra X chromosome); hemochromatosis (too much iron in the body); dysfunction of the pituitary gland; inflammatory diseases such as sarcoidosis; medications, especially hormones used to treat prostate cancer and corticosteroid drugs; chronic illness; chronic kidney failure; liver cirrhosis; stress, alcoholism, and obesity (especially abdominal obesity).

“Treat” as used herein refers to any type of treatment that imparts a benefit to a subject, including but not limited to delaying the onset or reducing the severity of at least one symptom in the subject

“Pharmaceutically acceptable” as used herein means that the compound or composition is suitable for administration to a subject to achieve the treatments described herein, without unduly deleterious side effects in light of the severity of the disease and necessity of the treatment.

### 1. Cells.

Cells used to carry out the present invention are, in general, live mammalian cells collected from a suitable donor. Donors are, in general, mammalian (e.g., human, dog, cat, rabbit, rat, mouse, monkey, chimpanzee, horse, pig, goat, sheep). The donor may be of the same species as the subject being treated, or of a different species. In some embodiments the donor may be the same subject undergoing treatment, where suitable cells were harvested from the subject and stored for subsequent use.

Cells are isolated from donors and cultured for microcapsule production as desired in accordance with techniques known in the art, See, e.g., Sanjay K. Agarwal et al., Leptin Antagonizes the Insulin-Like Growth Factor-I Augmentation of Steroidogenesis in Granulosa and Theca Cells of the Human Ovary, J. Clin Endocrinol Metab 84: 1072-1076 (1999); Jon C. Havelock et al., Ovarian granulosa cell lines, Molecular and Cellular Endocrinology 228, 67-78 (2004); Jessica K. Wickenheisser et al., Human ovarian theca cells in culture, Trends in Endocrinology & Metabolism 17, 65-71 (2006). In general, fresh tissue is divided by mincing, teasing, comminution and/or collagenase digestion. The desired cells are then isolated from contaminating cells and materials by washing, filtering, centrifuging or picking procedures, and optionally cultured and/or cryopreserved as desired prior to encapsulation.

Isolation and encapsulation of mammalian ovary cells, particularly ovarian theca cells, may be carried out as described in E. Opara et al., PCT Application WO 2012/121874 (Published Sep. 13, 2012), the contents of which is set forth further herein above and below.

Mammalian Leydig cells may be isolated in accordance with known techniques, including but not limited to those described in M. Machluf et al., Microencapsulation of Leydig Cells: A System for Testosterone Supplementation, Endocrinology 144, 4975 (2003).

Mammalian Sertoli cells may be isolated in accordance with known techniques, including but not limited to those described in M. Anway et al., Isolation of sertoli cells from adult rat testes: an approach to ex vivo studies of Sertoli cell function. Biol Reprod. 68:996-1002 (2003); P F Oliveira, et al., Influence of 5a-dihydrotestosterone and 17β-estradiol on human Sertoli cells metabolism. International Journal of Andrology. 08/2011; 34(6 Pt 2):e612-20 (2011); and L. Rato Et al., Metabolic modulation induced by oestradiol and DHT in immature rat Sertoli cells cultured in vitro, Bioscience Reports 32: 61-(9 (2011)

### 2. Microcapsule Production.

Encapsulation of live cells can be carried out in accordance with known techniques or variations thereof that will be apparent to those skilled in the art. See, e.g., U.S. Pat. Nos. 6,783,964 and 6,365,385 to Opara, the disclosures of which are incorporated by reference herein in their entirety.

Microcapsules useful in the present invention optionally, but in some embodiments preferably, have at least one semipermeable membrane surrounding a cell-containing interior. The semipermeable membrane permits the diffusion of nutrients, biologically active molecules and other selected products through the surface membrane and into the microcapsule core. The surface membrane contains pores of a size that determines the molecular weight cut-off of the membrane. The membrane pore size is chosen to allow the passage of estrogen, and in some embodiments progesterone, from within the capsule to the external environment, but to exclude the entry of host immune response factors (where the encapsulated cells are not autologous). Such a semipermeable membrane is typically formed from a polycation such as a polyamine (e.g., polylysine and/or polyomithine), as discussed further below.

In one non-limiting example embodiment of an encapsulation technique, U.S. Pat. No. 4,391,909 to Lim et at describes a method in which cells are suspended in sodium alginate in saline, and droplets containing cells are produced. Droplets of cell-containing alginate flow into calcium chloride in saline. The negatively charged alginate droplets bind calcium and form a calcium alginate gel. The microcapsules are washed in saline and incubated with poly-L-lysine or poly-L-ornithine (or combinations thereof); the positively charged poly-l-lysine and/or poly-L-ornithine displaces calcium ions and binds (ionic) negatively charged alginate, producing an outer poly-electrolyte semipermeable membrane. An exterior coating of sodium alginate may be added by washing the microcapsules with a solution of sodium alginate, which ionically bonds to the poly-L-lysine and/or poly-L-ornithine layer (this serves to reduce any inflammatory response that may be provoked in the subject by contact of the polycationic membrane to tissue). This technique produces what has been termed a “single-wall” microcapsule. A “double-wall” microcapsule can be produced by following the same procedure as for single-wall microcapsules, but prior to any incubation with sodium citrate, the microcapsules are again incubated with poly-l-lysine and sodium alginate.

In additional non-limiting examples of encapsulation methods, Chang et al., U.S. Pat. No. 5,084,350 discloses microcapsules enclosed in a larger matrix, where the microcapsules are liquefied once the microcapsules are within the larger matrix. Tsang et al., U.S. Pat. No. 4,663,286 discloses encapsulation using an alginate polymer, where the gel layer is cross-linked with a polycationic polymer such as polylysine, and a second layer formed using a second polycationic polymer (such as polyornithine); the second layer can then be coated by alginate. U.S. Pat. No. 5,762,959 to Soon-Shiong et al. discloses a microcapsule having a solid (non-chelated) alginate gel core of a defined ratio of calcium/barium alginates, with polymer material in the core. U.S. Pat. Nos. 5,801,033 and 5,573,934 to Hubbell et al. describe alginate/polylysine microspheres having a final polymeric coating (e.g., polyethylene glycol (PEG)); Sawhney et al., Biomaterials 13:863 (1991) describe alginate/polylysine microcapsules incorporating a graft copolymer of poly-l-lysine and polyethylene oxide on the microcapsule surface, to improve biocompatibility; U.S. Pat. No. 5,380,536 describes microcapsules with an outermost layer of water soluble non-ionic polymers such as polyethylene(oxide). U.S. Pat. No. 5,227,298 to Weber et al. describes a method for providing a second alginate gel coating to cells already coated with polylysine alginate; both alginate coatings are stabilized with polylysine. U.S. Pat. No. 5,578,314 to Weber et al. provides a method for microencapsulation using multiple coatings of purified alginate. U.S. Pat. No. 5,693,514 to Dorian et al. reports the use of a non-fibrogenic alginate, where the outer surface of the alginate coating is reacted with alkaline earth metal cations comprising calcium ions and/or magnesium ions, to form an alkaline earth metal alginate coating. The outer surface of the alginate coating is not reacted with polylysine. U.S. Pat. No. 5,846,530 to Soon-Shiong describes microcapsules containing cells that have been individually coated with polymerizable alginate, or polymerizable polycations such as polylysine, prior to encapsulation.

When desired, the alginate-polylysine microcapsules can be incubated in sodium citrate to solubilize any calcium alginate that has not reacted with poly-l-lysine, i.e., to solubilize the internal core of sodium alginate containing the cells, thus producing a microcapsule with a liquefied cell-containing core portion. See Lim and Sun, Science 210:908 (1980). Such microcapsules are referred to herein as having “chelated”, “hollow” or “liquid” cores.

When desired, the microcapsules may be treated or incubated with a physiologically acceptable salt such as sodium sulfate or like agents, in order to increase the durability of the microcapsule, while retaining or not unduly damaging the physiological responsiveness of the cells contained in the microcapsules. See, e.g., U.S. Pat. No. 6,783,964 to Opara.

One currently preferred method for the production of microcapsules is described in O. Khanna et al., Synthesis of multilayered alginate microcapsules for the sustained release of fibroblast growth factor-1 J. Biomed. Mater. Res. Part A: 95A: 632-640 (2010).

Microcapsules may be of any suitable size, such as from 10, 20 or 30 microns in diameter, up to 1000, 2000, or 5000 microns in diameter.

Microcapsules may contain any suitable amount of cell. For example, the theca cells are included in the microcapsules an amount of from 1,000 or 2,000 cells per microcapsule up to 1×10⁶, 1×10⁸, or 1×10⁹ cells per microcapsule.

When Sertoli and/or Leydig cells are included in the composition, they may be included in the same microcapsules or different capsules as the theca cells. Where included in the same microcapsules as the theca cells, they may be contained in the same compartment, or a different compartment, from the theca cells. For example, the microcapsules may include an auxiliary layer, with the theca cells in the core, with the sertoli cells in an auxiliary layer surrounding the theca cells, and Leydig cells in either the core, or the auxiliary layer (or both). In the alternative, the Sertoli cells may be in the core, the theca cells in the auxiliary layer, and the Leydig cells in either the core, the auxiliary layer around the core, or both. Sertoli and/or Leydig cells may be included in any suitable amount, for example, the same numbers as given above for theca cells, or one-half or one-third of those amounts. Where Leydig cells are included in addition to the theca cells, the number of theca cells may optionally be reduced, to for example one-half or one-third of those numbers given above, although the theca cells are preferably included in a treatment-effective amount.

While granulosa cells may be entirely excluded from the microcapsules, in some embodiments they may be included in small amounts (to facilitate the production of small amounts of estrogen, so long as they are not included in such amounts as to interfere with the predominant production of testosterone desired for the treatment of subjects with low testosterone. Thus in some embodiments, the granulosa cells may be included in the microcapsules in an amount less than 400, 600, 800 or 1,000 cells per microcapsule.

Microcapsules of the present invention may be administered after production, refrigerated and/or cryopreserved for subsequent use, and/or cultured for subsequent use, as desired. Microcapsules of the invention may be washed (e.g., in sterile physiological saline solution) prior to formulation and/or administration, as needed depending upon their manner of production.

### 3. Formulation and Administration.

Microcapsules of the present invention may be administered per se or formulated for administration by any suitable technique, such as by mixing with sterile physiological saline solution. Microcapsules of the present invention may be administered to subjects as a treatment for any condition in which estrogen replacement therapy is used. The microcapsules may be administered by any suitable technique, including but not limited to surgical implantation or injection (either of which may be carried out subcutaneously, intraperitoneally (for example, into the omentum), intramuscularly, or into any other suitable compartment. Dosage of cells administered can be determined in accordance with known techniques or variations thereof that will be apparent to those skilled in the art. For comparison, in the treatment of diabetes, the International Islet Transplant Registry has recommended transplants of at least 6,000 cells per kilogram of recipient body weight, to achieve euglycemia. In the present invention, the number of cells implanted will depend upon the age and condition of the subject, the particular disorder being treated, etc. In some embodiments of the present invention, from 1,000, 2,000 or 3,000 cells per kilogram of recipient body weight, up to 20,000, 40,000 or 60,000 cells per kilogram recipient body weight, are administered.

Subjects or patients to be treated by the methods of the present invention include subjects afflicted with, or at increased risk of, one or more of decreased libido, sadness or depression; lack of energy, lethargy or fatigue; risk of decreased height and/or loss of bone strength or density, fragile bones, decreased strength or loss of muscle mass, erectile dysfunction, shrinking testicles, numbness of testicles, infertility, decrease in ejaculate, decrease or less intense sexual response, increased body fat, hot flashes, swelling or tenderness of breast tissue, sleep apnea, insomnia, decrease in cognitive capacity (loss of memory and/or concentration), lower motivation, lower self-confidence, and decreased body hair.

The present invention is explained in greater detail in the following non-limiting Examples.

### EXAMPLE 1

### Isolation of Rat Ovaries

Postnatal day 21 Fischer 344 rats were injected with 1.5 mg/0.2 ml of 17β-estradiol (E2) dissolved in sesame oil, subcutaneously for three consecutive days. The rats were euthanized 24 h after the last injection, ovaries were excised and endocrine cells were isolated as described in Example 2:

### EXAMPLE 2

### Cell Isolation and Purification

The endocrine cells were isolated from ovaries of E2-primed immature rats according to Li and Hearn (J. Biochem. Biophys. Methods 45, 169-181 (2000), Ovaries collected in ice cold medium 199 (M199) containing HEPES (25 mM), 1 mg/ml bovine serum albumin (BSA), L-glutamine (2 mM), penicillin (10,000 IU/ml), streptomycin (10,000 μg/ml), and amphotericin B (25μg/ml). After cleaning the extraneous tissues, the ovaries were washed twice with ice cold M199 and then punctured gently with 27G syringe needles in order to release the loosely packed granulosa from the follicles; cells thus collected were kept on ice. The remaining ovaries were chopped into fine pieces of ˜0.25 mm² and the cells released during this process were collected and kept on ice separately. The pieces of ovaries were then incubated with collagenase (2 mg/ml) and DNase (10 μg/ml) in M199 for 90 min with occasional mixing. The enzyme-digested pieces were dispersed using a Pasteur pipette to obtain a single cell suspension and collected and stored on ice as a separate fraction, Cells from different fractions collected above were purified as per Magoffin and Erickson (Endocrinology 122, 2345-2347 (1988)). Briefly, the cells were loaded on top of a discontinuous percoll gradient (44% in the bottom, d=1.055 percoll (specific gravity adjusted to 1.055) in the middle and 20% on the top) and centrifuged at 400×g for 20 minutes at 4° C. Cells from the first interphase (between 20% and d=1.055 layers) were recovered as granulosa cells and those from the second interphase (between d=1.055 and 44% layers) were collected as theca cells (not shown). The viability of the cells was checked using the trypan blue method and was in the 85-95% range. The purity of each cell type was assessed by flow cytometric analysis using cell-specific markers.

### EXAMPLE 3

### Cells Analyzed Using Flow Cytometry

A fraction of the cells (5×10⁶ cells/cell type) purified using the discontinuous percoll gradient was fixed in 3.7% formaldehyde for 15 minutes.

To verify the purity of the cell types isolated from the rat ovaries, the cells were stained with cell-specific markers and quantified by flow cytometry. Cells from different interphases (not shown) were incubated with primary antibodies. Antibody for CYP19 (mouse snit-CYP19; Abbiotech; cat. 250549) and FITC-conjugated secondary antibody were used to detect the granulosa cells. Antibody for CYP17A1 (goat anti-CYP17A1; Santa Cruz Biotechnology; cat. sc-46085) and PerCP Cy5.5-conjugated donkey anti-goat IgG secondary antibody were used to detect the theca cells. Cells were incubated with the appropriate primary antibody for 1 h. Unbound antibodies were then washed off and the cells were incubated with the appropriate secondary antibody for 1 h. After washing off the unbound secondary antibodies, cells were analyzed using flow cytometry. The flow cytometric analysis revealed that 74.15% of the cells recovered from the first interphase in the percoll gradient stained positive for CYP19 (not shown) and 69.91% of the cells obtained from the second interphase stained for CYP17A1 (not shown). Cells incubated with only secondary antibodies were used as control.

### EXAMPLE 4

### Culture of Granulosa and Theca Cells

Purified granulosa and theca cells were separately incubated at 37° C. under an atmosphere of 5% CO₂ in humidified air in T175 flasks (Corning, Corning Inc., NY, USA) cultured for 24 h in McCoy's 5A medium supplement with L-glutamine (2 mM), penicillin (10,000 IU/ml), streptomycin (10,000 μg/ml), amphotericin B (25 μg/ml) and 10% FBS. The medium for granulosa cells was replaced with granulosa growth medium (McCoy's 5A with L-glutamine mM), BSA (1 mg/ml), penicillin (10,000 IU/ml), streptomycin (10,000 μg/ml), and amphotericin B (25 μg/ml), 200 ng/ml oFSH, 100 nM E2 and 10 nM IGF-I) and cultured for an additional 72 h. Similarly, the theca cells were grown for another 72 h in theca growth medium (McCoy's 5A medium supplemented with L-glutamine (2 mM), BSA (1 mg/ml), penicillin (10,000 IU/ml), streptomycin (10,000 μg/ml), amphotericin B (25 μg/ml), 100 ng/ml oLH; 10 nM IGF-I).

### EXAMPLE 5

### Immuno-fluorescence Staining

Each cell type was cultured on chamber slides in respective growth medium and screened for the expression of essential cellular components for steroidogenesis. After fixing the cells in 3.7% formaldehyde for 15 minutes, cells were washed with PBS and blocked with PBS with BSA (1%). The monolayer was then incubated with primary antibodies overnight at 4° C. Granulosa cells were incubated with rabbit anti-FSHR (Santa Cruz Biotechnology; cat. no. sc-13935) and mouse anit-CYP19 (Abbiotech; cat. no. 250549). Similarly theca cells were incubated with rabbit anti-LHR (Santa Cruz Biotechnology; cat. no. se-25828) and goat anti-CYP17A1 (Santa Cruz Biotechnology; cat. no. se-46085). After overnight incubation with primary antibodies, the slides were washed with PBS and incubated with secondary antibodies for 2 h at 4° C. The unbound secondary antibodies were washed away and the nucleus was counterstained with DAPI and cover slips were mounted. The images were acquired using a fluorescence microscope and composite images were made with the help of Image-Pro plus software version 6.3.1.542.

While theca cells stained positive for LH-receptor (LHR) and CYP17A1 (data not shown), granulosa cells showed positive for FSH-receptor (FSHR) and CYP19 (data not shown).

### EXAMPLE 6

### Granulosa Cells and Theca Cells Encapsulated Separately

Cultured cells were encapsulated separately by extrusion through a multi-nozzle extruder in 1 to 3% (w/v) ultrapure low viscosity high-mannuronic (LVM) alginate solution into calcium chloride solution for 5 to 15 minutes (for cross-linking) to produce microcapsules of approximately 300 to 600 micron diameter. All the encapsulation and washing steps are carried out at room temperature. Granulosa cell-containing microcapsules and theca cell-containing microcapsules were then combined together with one another in equal parts, co-cultured together in separate chambers of culture inserts in 24-well plates in McCoy's 5A medium supplemented with penicillin/streptomycin (100 IU/ml & 100 μg/ml, respectively), amphotericin B (0.25 μg/ml) and fetal bovine serum (10%) at 37° C. and 5% CO₂. The viability and 17β-estradiol production as discussed below was evaluated periodically for 30 days.

The microcapsules received 50 ng/ml follicle-stimulating hormone (FSH) and 50 ng/ml luteinizing hormone (LH) in long-term cultures. LH treatment increased the expression of CYP17A1 (17, 20 lyase) in theca cells and FSH treatment increased the expression of CYP19 (aromatase) in granulosa cells in vitro (not shown), which improves the steroidogenic potency of these cells. Encapsulation distributed cells evenly in the alginate microcapsules (not shown). It was noted that optimum cell density is an important factor for configuration and structure of the microcapsule, which was approximately 1,000 to 10,000 cells per microcapsule.

Encapsulated cells had sustained viability during the long-term culture up to day 30 (data not shown). The number of non-viable cells increased in the course of long-term culture.

Note that granulosa cell-containing microcapsules co-cultured with theca cell-containing microcapsules produced significantly higher levels of E2 than either cultured individually (data not shown).

In addition, co-culture of granulosa cell-containing microcapsules with theca cell-containing microcapsules secreted increased levels of E2 in response to FSH and LH in the long-term culture in vitro (data not shown).

These data show that ovarian endocrine cells encapsulated in alginate hydrogel microcapsules showed both long-term survival and bioactivity in vitro. With the encapsulation technique we were able to demonstrate that the endocrine unit of ovaries could be recapitulated ex vivo.

In additional experiments, portions of microcapsules were cultured in the presence of FSH (100 ng/ml) and LH (100 ng/ml) for about 30 days and the culture media were collected every alternate day to test the secretion of sex steroids. The levels of 17β-estradiol and progesterone in the culture media were quantified using ELISA kits. 17β-estradiol in culture media was measured with an ELISA kit from Enzo Life Sciences (cat. No. ADI-901-008). The progesterone levels in cell culture media were measured using the ELISA kit from Enzo Life Sciences (cat. no. ADI-901-011). The levels of 17β-estradiol and progesterone were quantified according to the manufacturer's instructions and corrected for their dilutions.

When granulosa cell-containing microcapsules or theca cell-containing microcapsules were incubated separately, there were no significant increases in the production of 17β-estradiol. In the same experiments, the progesterone levels reached 1.3 and 0.8 ng/ml at days 4 and 6, respectively (data not shown).

When granulosa cell-containing microcapsules and theca cell-containing microcapsules were co-cultured, the 17β-estradiol level reached ˜20 pg/ml at day 18 and the progesterone level peaked at ˜1.5 ng/ml at day 26 (data not shown).

### EXAMPLE 7

### Granulosa Cells and Theca Cells Encapsulated Together

This example is carried out in like manner as Example 6 above, except that the granulosa and theca cells are mixed together in essentially equal amounts prior to extrusion, so that the two are encapsulated together.

A portion of microcapsules were cultured in the presence of FSH (100 ng/ml) and LH (100 ng/ml) for about 30 days and the culture media were collected every alternate day to test the secretion of sex steroids. The levels of 17β-estradiol and progesterone in the culture media were quantified using ELISA kits. 17β-Estradiol in culture media was measured with an ELISA kit from Enzo Life Sciences (cat. No. ADI-901-008). The progesterone levels in cell culture media were measured using the ELISA kit from Enzo Life Sciences (cat. no. ADI-901-011). The levels of 17β-estradiol and progesterone were quantified according to the manufacturer's instructions and corrected for their dilutions. Encapsulated cells responded to the gonadotropins from day 2 onward. The 17β-estradiol levels were approximately 5-fold higher by day 25, when compared to basal levels, and the progesterone levels were approximately 2 fold higher when compared to basal levels (not shown).

### EXAMPLE 8

### Porcine Bone Marrow Stromal Cells Encapsulated Together in Layers

Two layer microcapsules were produced in accordance with the technique described in O. Khanna et al., J. Biomed. Mater. Res. Part A 95A: 632-640 (2010). Briefly, porcine bone marrow stromal cells (pBMSC) were cultured in DMEM supplemented with penicillin/streptomycin (100 IU/ml & 100 μg/ml, respectively), amphotericin B (0.25 μg/ml), fetal bovine serum (10%) at 37° C. and 5% CO₂ and tagged with vital fluorescent probe CellTracker green and CellTracker orange (invitrogen). pBMSC probed with CellTracker green were encapsulated in 1-2% low viscosity high-mannuronic (LVM) alginate by extrusion through a multi-nozzle extruder into a calcium chloride solution. The microcapsules were then suspended with a 0.05 to 0.2% poly-L-ornithine solution for about 5 to 30 minutes at 4° C. to create the permselective membrane layer. The coated microcapsules were then coated with a second layer of alginate, which was 0.5 to 2% (w/v) low viscosity high-glucoronic alginate (LVG) containing CellTracker orange-probed pBMSC. About 1,000 to 10,000 cells are included in each layer of the capsule.

### EXAMPLE 9

### Granulosa Cells and Theca Cells Encapsulated In a Two layer Microcapsule

Granulosa cells were encapsulated in 1.5% (w/v) LVM and coated with poly-L-ornithine (PLO) (0.1% w/v) for 20 minutes. The PLO-coated microcapsules were then mixed with theca cells suspended in 1.5% (w/v) LVM and encapsulated again using the micro-fluidic device (not shown) in order to obtain multi-layered microcapsules, which resemble the structural architecture of native follicles (not shown; referred to as multi-layered microcapsules).

A portion of microcapsules were cultured in the presence of FSH (100 ng/ml) and LH (100 ng/ml) for about 30 days and the culture media were collected every alternate day to test the secretion of sex steroids. The levels of 17β-estradiol and progesterone in the culture media were quantified using ELISA kits. 17β-estradiol in culture media was measured with an ELISA kit from Enzo Life Sciences (cat. No. ADI-901-008). The progesterone levels in cell culture media were measured using the ELISA kit from Enzo Life Sciences (cat. no. ADI-901-011). The levels of 1713-estradiol and progesterone were quantified according to the manufacturer's instructions and corrected for their dilutions. There was a ten-fold increase in the 17β-estradiol by day 25 and progesterone levels were approximately 2 fold higher when compared to basal levels (data not shown).

To demonstrate the differential compartmentalization of different cell types in the multi-layered microcapsules, the granulosa cells were pre-stained with Cell Tracker green (Invitrogen, cat. No. C2925) and the theca cells were pre-stained with Cell-tracker Orange (Invitrogen, cat. No. C2927), prior to the synthesis of the multi-layered microcapsules. The multi-layered microcapsules were imaged using a confocal microscope (Zeiss LSM510).

### EXAMPLE 10

### Viability of Encapsulated Ovarian Endocrine Cells

The viability of the encapsulate cells were assessed using live dead analysis. A portion of microcapsules from Examples 6, 7, 8, and 9 were cultured in the presence of FSH (100 ng/ml) and LH (100 ng/ml) for about 30 days and the culture media were collected approximately every third day to test the viability of the encapsulated cells. At the designated times, encapsulated cells were transferred to a 24-well plate and incubated with 25 μM CFDA SE (carboxyfluorescein diacetate, succinimidyl ester) (Invitrogen, cat. no. V12883) in serum-free medium for 15 minutes at 37° C. under an atmosphere of 5% CO₂ in humidified air. Then the CFDA containing medium was replace with medium containing 10% FBS and incubated again under the above-mentioned conditions for an additional 30 min. The serum-containing medium was then replace with 50 μg/ml of propidium iodide (PI) (Invitrogen, cat. no. V12883) and incubated at room temperature for 2 min and the microcapsules were washed ₜo remove excess PI. The microcapsules were then observed under an inverted fluorescence microscope and imaged. The number of live and dead cells was analyzed from the acquired composite image using Image-Pro plus software version 6.3.1.542.

Note: live cells cleave the ester group of membrane permeable non-fluorescent CFDA and convert it into non-permeable-green fluorescent FDA, which gets trapped inside viable cells. On the other hand, dead cells have a compromised membrane whereby propidium penetrates into the nucleus and stains the DNA red. The periodical live/dead analysis revealed the encapsulated ovarian endocrine cells had a sustained viability throughout the period of long-term culture (not shown).

### EXAMPLE 11

### Encapsulation of Theca Cells Only for Treatment of Low Testostereone

This example is carried out in like manner as described in Example 9 above, except that the granulosa cells are replaced with theca cells in the initial step to produce a multi-layer construct containing theca cells in both layers.

The foregoing is illustrative of the present invention, and is not to be construed as limiting thereof. The invention is defined by the following claims, with equivalents of the claims to be included therein.

1. A pharmaceutical composition for treating low testosterone, said composition comprising microcapsules, said microcapsules containing live mammalian ovary cells, said ovary cells consisting essentially of ovarian theca cells in a treatment-effective amount;
said microcapsules optionally containing live mammalian Sertoli cells; and
and said microcapsules optionally containing live mammalian Leydig cells.

2. A composition of claim 1, wherein said sertoli cells, said Leydig cells and said theca cells are contained in separate microcapsules in said composition.

3. The composition of claim 1, wherein said sertoli cells, said Leydig cells and said theca cells are contained together in the same microcapsules in said composition.

4. The composition of claim 3, wherein said microcapsules comprise a core and an auxiliary layer surrounding said core;
said core containing one of said Sertoli cells and said theca cells and said auxiliary layer containing the other of said Sertoli cells and said theca cells;
and with said core and/or said auxiliary layer optionally further containing said Leydig cells.

5. The composition of claim 4, said microcapsules further comprising a first semipermeable layer between said core and said auxiliary layer.

6. The composition of claim 4, said microcapsules further comprising a second semipermeable layer surrounding said auxiliary layer.

7. The composition of claim 4, said microcapsules further comprising an external polysaccharide layer surrounding said second semipermeable layer.

8. The composition of claim 4, wherein said semipermeable layers are formed of a polycation.

9. The composition of claim 8, wherein said polycation is a polyamine.

10. The composition of claim 1, wherein said composition is free of oocytes.

11. The composition of claim 1, wherein said microcapsules comprise a hydrogel.

12. The composition of claim 11, wherein said hydrogel comprises a polysaccharide hydrogel.

13. The composition of claim 1, wherein said microcapsules are from 10 microns in diameter, up to 5000 microns in diameter.

14. The composition of claim 1, wherein said theca cells are included in said microcapsules in an amount of from 1,000 cells per microcapsule up to 1×10⁹ cells per microcapsule.

15. A method of administering testosterone to a male subject in need thereof, comprising administering said subject a composition of claim 1 in a treatment-effective amount.

16. The method of claim 15, wherein said administering step is carried out by parenteral injection.

17. (canceled)
